(19) United States
(12) Patent Application Publication
(10) Pub. No.: US 2011/0237537 A1
(43) Pub. Date: Sep. 29, 2011

(54) METHODS FOR ASSESSMENT AND TREATMENT OF MOOD DISORDERS VIA SINGLE NUCLEOTIDE POLYMORPHISMS ANALYSIS

(76) Inventor: Jay L. Lombard, New City, NY (US)

(21) Appl. No.: 13/074,967

(22) Filed: Mar. 29, 2011

Related U.S. Application Data
(63) Continuation-in-part of application No. 12/790,262, filed on May 28, 2010.
(60) Provisional application No. 61/217,338, filed on May 29, 2009.
(60) Provisional application No. 61/325,098, filed on Apr. 16, 2010.
(60) Provisional application No. 61/410,523, filed on Nov. 5, 2010.
(60) Provisional application No. 61/321,065, filed on Apr. 5, 2010.
(60) Provisional application No. 61/321,281, filed on Apr. 6, 2010.

(51) Int. Cl. A61K 31/7076; A61K 31/554; A61K 31/4015; A61K 31/4184; A61K 31/519; A61K 31/5513; A61P 25/00; A61P 25/18; C12Q 1/68; C40B 30/04

(52) U.S. Cl. 514/46; 514/211.13; 514/423; 514/381; 514/250; 514/220; 435/6.11; 506/9

(57) ABSTRACT

Described herein are assays, kits and methods for treating mood disorders by testing for one or more polymorphisms in a specific group of genes and for analyzing the results of polymorphism testing; the genes included may converge in one or more signaling pathways, and may be epigenetic. The genes are included based on the relationships of the proteins encoded by the genes in the context of particular signaling pathways and provide a diagnostically relevant nexus. Also described herein are methods of presenting the data collected by the screen, including methods of delivering interpretive comments and/or treatment guidance based on the results of the genetic screening either individually or based on the genetic composition of particular clusters of genes which may be related to each other. Importantly, drugs which modulate these genetic disturbances are described for targeted therapeutic use based upon companion diagnostic method.

### CROSS REFERENCE TO RELATED APPLICATIONS

This patent application claims priority as a continuation-in-part of U.S. patent application Ser. No. 12/790,262, filed on May 28, 2010, titled “METHOD FOR ASSESSMENT AND TREATMENT OF DEPRESSION VIA UTILIZATION OF SINGLE. NUCLEOTIDE POLYMORPHISMS ANALYSIS,” which claims priority to U.S. Provisional Patent Application No. 61/217,338, filed on May 29, 2009, titled “SYSTEM AND METHOD FOR DIAGNOSIS AND TREATMENT OF COMMON MENTAL HEALTH COMPLAINTS,” and U.S. Provisional Patent Application 61/325,098, filed on Apr. 16, 2010, titled “MODULATION OF SEROTONIN REUPTAKE BASED ON GENOTYPE TO TREAT DEPRESSION,”

This patent application also claims priority to U.S. Provisional Patent Application Nos. 61/410,523, filed on Nov. 5, 2010, titled “TREATMENT RESISTANT DEPRESSION DIAGNOSTIC TEST REPORT;” 61/321,065, filed on Apr. 5, 2010, titled “MEDICAL FOODS FOR THE TREATMENT OF DEPRESSION AND NEURODEGENERATIVE DISORDERS;” and 61/321,281, filed on Apr. 6, 2010, titled “TREATMENT OF ALZHEIMERS DISEASE BY MODULATION OF ANTIMICROBIAL PEPTIDES.” All of the patent applications mentioned above are herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The devices, methods, and systems described herein relate to the diagnosis and treatment of mood disorders, and particularly to the treatment of depression based on the determination of genetic predispositions related to common neurotransmitter pathway based polymorphisms, including serotonin, glutamate and dopamine. The screens described herein are gathered into a screen based on a clinically and therapeutically relevant nexus.

### BACKGROUND OF THE INVENTION

Between 5-10% of adults worldwide suffer from depression.

The economic costs to society and the personal costs to individuals and families, associated with depression are enormous. Within a 15-month period after having been diagnosed with depression, sufferers are four times more likely to die as those who do not have depression. Almost 60% of suicides have their roots in major depression, and 15% of those admitted to a psychiatric hospital for depression eventually kill themselves. In the U.S. alone, the estimated economic costs for depression in 1990 exceeded $44 billion. The World Health Organization estimates that major depression is the fourth most important cause worldwide of loss in disability-adjusted life years, and will be the second most important cause by 2020.

A variety of pharmacologic agents are available for the treatment of depression. Significant success has been achieved through the use of serotonin reuptake inhibitors (SRIs), norepinephrine reuptake inhibitors (NERIs), combined serotonin-norepinephrine reuptake inhibitors (SNRIs), monoamine oxidase inhibitors (NERIs), phosphodiesterase-4 (PDE4) inhibitors or other compounds. However, even with these options available, many patients fail to respond, or respond only partially to treatment. Additionally, many of these agents show delayed onset of activity, so that patients are required to undergo treatment for weeks or months before receiving benefits. Most currently available antidepressants take 2-3 weeks or more to elicit a response.

Traditional therapies can also have significant side effects. For example, more than a third of patients taking SRIs experience sexual dysfunction. Other problematic side effects include gastrointestinal disturbances, often manifested as nausea and occasional vomiting, agitation, insomnia, weight gain, onset of diabetes.

Thus, there remains a need for the development of improved therapies for the treatment of depression and/or other mood disorders.

In the clinic, 40-50% of depressed patients who are initially prescribed antidepressant therapy do not experience a timely remission of depression symptoms. This group typifies treatment-refractory depression, that is, a failure to demonstrate an “adequate” response to an “adequate” treatment trial (sufficient intensity of treatment for sufficient duration). Moreover, about 20-30% of depressed patients remain partially or totally resistant to pharmacological treatment.

This is increasing evidence implicating the role of neurotransmitters depression, in particular the monoamines serotonin, noradrenaline, dopamine, as well as the excitatory amino acid glutamate. Many of the tricylic antidepressants (TCAs), selective serotonin re-uptake inhibitors (SSRIs) and monoamine oxidase inhibitors (MAOIs) effective in the treatment of depression increase the availability of the catecholamines (noradrenaline and dopamine) and indolamines (serotonin) in the central nervous system (CNS). The clinical efficacy of these agents has given rise to the catecholamine-indolamine hypothesis of depression. This theory postulates that a certain level of amines and/or receptor sensitivity to catecholamines functions to generate a normal mood. Receptor insensitivity, a depletion of monoamines, or a decrease in their release, synthesis or storage has been postulated to lead to depression. Other agents are also increasingly being used to treat depression, including mood stabilizers and anti psychotics. The increasing choices and complexity of decision making when treating depression is reinforced by the differences in activity on neurotransmitter systems these class agents have.

Although previous work has suggested the use of certain SNPs to diagnose depression (see, for example, US 2008/0299125 to Hinds et al., US 2008/0199866 to Akil et al., US 2008/0268436 to Duan et al., US 2006/0160119 to Turner et al., US 2008/018076 to Chissoe, and US 2008/0118918 to Licinio et al.), the systems, assays and methods described herein are based on the discovery that the behavioral phenotypes of gene expression can be understood and interpreted in terms of the net effect of these particular genes on specific neurotransmitter based brain synaptic pathways. Specifically, the inventor has recognized that genotyping relevant neurotransmitter based pathways will further instruct on choosing between agents with distinct and divergent pharmacological activity.

### SUMMARY OF THE INVENTION

We herein postulate herein that depression subtypes are based upon imbalances of specific neurotransmitter pathways in the brain. Certain subtypes of depression are associated with predominant neurotransmitter imbalances, leading to specific phenomenological behavioral states. Thus, a clinician will be able to ascertain a specific subtype of depression by analyzing both the behavioral and genetic patterns of individuals with a mood disorder. The primary neurotransmitter based genes include serotonin pathway related genes, calcium mediated glutamate related genes, dopamine pathway related genes and methylation and metabolism genes.

As used herein, the term “mood disorder” may include any number of disorders, including, but not limited to: major depression, bipolar disease, psychotic disorders, childhood disorders, geriatric disorders, anxiety disorders, PTSD, and the like.

A screen for a cluster of genes is claimed which may include examining for polymorphisms of: CACNA1C, FKBP5, SERT, DRD2, BDNF, MTHF and COMT. Treatments are also claimed based upon identification of these polymorphisms.

In general, an SNP indicator indicates the presence or absence of an SNP from a tissue sample. The SNP indicator may be based on (or part of) a screening test, such as a genetic screen (e.g., using a PCR-based test) to determine if the SNP is present within the DNA of a particular patient's tissue sample being examined. Any appropriate test for the individual SNP or a pooled test for multiple SNPs may be used as part of the methods, kits, assays and systems described herein. As mentioned, the SNP indicators comprise one or more PCR-based assays. An SNP indicator may include a report (e.g., visual, oral, printed, electronic, or the like), and may indicate the presence or absence of the particular SNP. The SNP indicator may indicate if the SNP is homozygous or heterozygous.

For example, in some variations, the SNP indicator indicates an SNP that alters the function or expression of the Serotonin transporter genes in the serotonin metabolism pathway. In some variations, the SNP indicator indicates an SNP that alters the function or expression of the MTHF, COMT pathways or DRD2 genes in the dopamine metabolism pathway. In some variations, the SNP indicator indicates an SNP that alters the function or expression of the CACNA1C genes in the glutamate metabolism pathway. In some variations, the SNP indicator indicates an SNP that alters the function of FKBP5 genes in the hypothalamic pituitary adrenal axis.

The panel assay may include: a plurality of SNP indicators that collectively indicate the presence or absence of one or more SNP that alters the function or expression of a gene from each of the serotonin metabolism pathway, the dopamine metabolism pathway, the glutamate metabolism pathway, and the hypothalamic pituitary adrenal axis; and an interpretive comment indicating the effect of any identified SNPs.

For example, described herein are panel assays to determine the presence of SNPs that alter the function or expression of a gene from each of the serotonin metabolism pathway, the dopamine metabolism pathway, the glutamate metabolism pathway, and the drug metabolism pathway, in some variations, the panel assay also includes a report with one or more interpretive comments indicating the effect of any identified SNPs on the regulation of these pathways. The panel assay may also include an interpretive comment suggesting a treatment based on identified SNPs.

The SNP indicator may indicate an SNP that alters the function or expression of the SERT gene in the serotonin metabolism pathway; the DRD2 genes in the dopamine metabolism pathway; the CACNA1C gene in the glutamate metabolism pathway; genes that regulate methylatoin and/or drug metabolism, including: MTHFR, COMT and/or CYP2D6. Any appropriate method for testing for the SNP indicators described herein may be used, including PCR-based assays.

The assay may also include one or more an interpretive comment suggesting a treatment based on identified SNPs. For example, the SNP indicator may indicate an SNP that alters the function or expression of the SERT related genes in the serotonin metabolism pathway. The SNP indicator may indicate an SNP that alters the function or expression of the MTHF, or COMT genes in the methylation pathway; the SNP indicator may indicate an SNP that alters the function or expression of the CACNA1C genes in the glutamate metabolism pathway. The SNP indicator may indicate an SNP that alters the function or expression of the FKBP5 genes or BDNF in the hypothalamic pituitary adrenal axis.

Also described herein are mood disorder panel assays to guide therapeutic treatment by determining the presence of SNPs that alter the function or expression of: the SERT gene; one or more of: DRD2, MTHF, COMT genes; one or more of: CACNA1C; and one or more of: MTHFR, COMT and/or CYP2D6.

In some variations the mood disorder panel assay to determine the presence of SINPs that contribute to a mood disorder comprises: a plurality of SNP Indicators that collectively indicate the presence or absence of one or more SNP that alters the function or expression of a gene from each of the serotonin metabolism pathway, the dopamine metabolism pathway, the glutamate metabolism pathway, and the hypothalamic pituitary adrenal axis; and an interpretive comment suggesting a treatment based on the identified SNPs.

Also described herein are methods of treating a patient for a mood disorder comprising: determining the genotype of a polymorphism in each of the SERT, BDNF, CACNA1C, MTHFR/COMT and DRD2 genes; advising a treatment based upon the results of said testing.

In some variations a method of treating a patient for a mood disorder comprise: determining the genotype of a polymorphism in each of the SERT, BDNF, CACNA1C, MTHFR/COMT and DRD2 genes; advising a treatment based upon the results of said testing wherein the treatment comprises: prescribing at least one of tianeptine and other SSRE for patients having a SERT short allele; prescribing at least one of Aniracetam and Nefiracetam for patients having the Val66Met form of BDNF; prescribing at least one of a calcium channel antagonists, an L-type voltage gated calcium channel agonist, and a member of the ARB class of drugs, and Candesartan for patients having either the rs1006737 or the rs1006737 variant of CACNA1C; prescribing at least one of a methylating agent, MTHF, S adenosylmethionine, a dopamine agonists, a MAO inhibitor, and a stimulant, for patients having either the C677T MTHFR variant or the 158val/val allele of the COMT gene; and prescribing at least one of an atypical neuroleptics which preferentially inhibits 5HT2A over DRD2 and Clozaril for patients having the −141C Ins/Del.

In some variations the method of treating a patient for a mood disorder comprises: determining the genotype of a polymorphism in each of the SERT, BDNF, CACNA1C, MTHFR/COMT and DRD2 genes; providing a treatment based upon the results of said testing, wherein the treatment comprises: prescribing at least one of tianeptine and other SSRE for patients having a SERT short allele; prescribing at least one of Aniracetam and Nefiracetam for patients having the Val66Met form of BDNF; prescribing at least one of a calcium channel antagonists, an L-type voltage gated calcium channel agonist, and a member of the ARB class of drugs, and Candesartan for patients having either the rs1006737 or the rs1006737 variant of CACNA1C; prescribing at least one of a methylating agent, MTHF, S adenosylmethionine, a dopamine agonists, a MAO inhibitor, and a stimulant, for patients having either the C677T MTHFR variant or the 158val/val allele of the COMT gene; and prescribing at least one of an atypical neuroleptics which preferentially inhibits 5HT2A over DRD2 and Clozaril for patients having the −141C Ins/Del.

As mentioned above, the assay may also include an interpretive comment suggesting a treatment based on identified SNPs.

### BRIEF DESCRIPTION OF THE DRAWINGS

FIG. 1 is a flowchart illustrating one variation of a method for determining a net or predicted net effect of one or more SNPs.

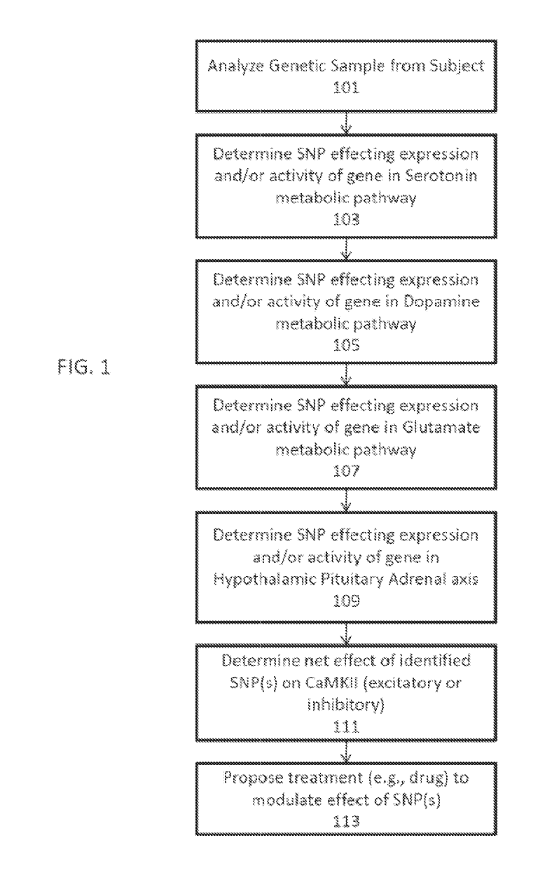

FIG. 2 is a table listing the pathways (e.g., serotonin, dopamine, glutamate, drug metabolism), genes tested in each pathway for polymorphisms, possible results (polymorphisms), interpretive comments and examples of therapies that may be applied guided by these results.

FIG. 3 is a chart indicating exemplary therapeutics that may be advisable based on particular SNP results.

### DETAILED DESCRIPTION OF THE INVENTION

Genes associated with neurotransmitter pathways are abnormal in patients with clinical depression. For instance, genes which regulate serotonin pathways, including genes coding for receptors, metabolism and reuptake mechanisms, are associated with depression. Furthermore, other genetic-neurotransmitter pathways, including dopamine, norepinephrine and glutamate are associated with depression. The heterogenous nature of these results suggests that depression as a disorder is itself heterogenous. By analyzing depression from a single nucleotide polymorphism based gene analysis, subtypes of depression can be differentiated and diagnosed. Accurate subtype depression based upon single nucleotide polymorphism is novel and previously undisclosed. Further, the employment of such analysis will allow mental health professionals who treat individuals with depression with more specific and targeted interventions.

Depression may be better dissected using paradigms that assess how specific genes associate with component features of depression. This approach reveals gene influences on trait components of depression and, may help identify depression subpopulations that can benefit from more targeted pharmacotherapy.

Many people complain of depressive symptoms, but despite the commonality of these complaints, there is significant biochemical heterogeneity regarding the etiology, phenomenology and treatment of mood disorders. This biochemical heterogeneity is evidenced by, inter alia, the occurrence of single nucleotide polymorphisms (SNPs) in genes involved with neurotransmitter activity related to depression. Subtle genomic variations create the chemistry that underlies subtypes of depression.

As an example, a single nucleotide polymorphism in the gene that regulates dopamine can be associated with altered neurotransmitter binding and differential drug effect in the brain. Patients with a dopamine based SNP thus differ not only in their symptoms but their response to therapies as well.

Based upon the recognition of SNP associated biochemical and symptomatic heterogeneity, a mood complaint such as depression can either be a consequence of a genetic defect that effects serotonin metabolism, but also can be a consequence of an SNP associated genetic defect in a gene that regulates dopamine, or some other neurotransmitter. As a similar example, depression can be etiologically associated with a SNP in glutamate in one individual, and with a SNP related to dopamine or norepinephrine in another.

The recognition of the distinction in the genetic and biochemical heterogeneity related to the expression of subtypes of depression has important therapeutic implications. Frequently, an individual with a mood disturbance does not respond favorably to a specific class of therapeutic agents but may respond to a different class of therapeutic agents. As an example, an individual who is experiencing depression due to a specific SNP related dopamine metabolism defect will not respond or respond less favorably to a serotonin modulating agent. In clinical practice, this can happen when a psychiatrist treats a patient with depression who possesses a SNP associated with a dopamine related defect with a serotonin modulating drug like sertraline paroxetine instead of a dopamine modulating drug such as buproprion or Aripirazole. In these instances, the drug may produce a worsening of symptoms instead of improving them.

Conversely, an individual with a SNP associated with serotonin metabolism may respond less favorably to a dopamine modulating agent. Unfortunately, psychiatrists administer medications for depression solely on a trial and error basis. The lack of diagnostic specificity frequently leads to ineffective treatments, substantial side effects or a delay in the proper treatment.

Thus, a common problem in the management of mood disorders is a lack of diagnostic specificity and/or treatments which are not coupled to the unique neurotransmitter disturbance related to depression. Provided herein is a method of using the analysis of an individual's SNPs related to neurotransmitter function as an aid to diagnosis and choice of therapeutic treatment. It is an object of this description to set forth the specific genomic sites that are causally associated with the biochemical and neurochemical abnormalities associated with depression. The ability to accurately identify SNPS related to neurotransmitter imbalances and depression subtypes represents an advance in the field of mental health.

The methods regarding the employment of genes related to neurochemical imbalances are broadly applied to the genes involved in at least the following pathways: Serotonin, dopamine, glutamate and the hypothalamic pituitary adrenal axis. Specific genes within these categories are described in the paragraphs herein but are not limited to this disclosure. Thus, while the present invention describes polymorphisms in specific serotonin pathways, it is recognized that other polymorphisms in the serotonin pathway are contemplated as within the scope of this disclosure.

Genomic polymorphisms in the following glutamate related pathways are associated with depression: CACNAC gene.

Genomic polymorphisms in the following dopamine related pathways are associated with depression: DRD2.

Genomic polymorphisms associated with methylation pathway: MTHFR gene and COMT gene.

Genomic polymorphisms in the following serotonin related pathways are associated with depression: the serotonin transporter gene.

The hypothalamic pituitary adrenal axis has been recognized as a critical region in the stress response as well as in the pathophysiology of depression.

The FKBP5 gene and BDNF gene are related to the stress response, and the hypothalamic pituitary adrenal pathway.

These gene categories are associated with depression subendophenotypes, the analysis of which through single nucleotide polymorphisms is applied to provide a more accurate and specific therapeutic intervention based upon the neurochemical consequences of these genetic polymorphisms.

A summary of the neurochemical assessment based upon the analysis of single nucleotide polymorphisms is subsequently provided below.

### Glutamate Pathway Associated Genes

CACNA1C (rs1006737) G>A and (rs10848635) T>A

The calcium ion is one of the most versatile, ancient, and universal of biological signaling molecules, know o regulate physiological systems at every level from membrane potential and ion transporters to kinases and transcription factors. Disruptions of intracellular calcium homeostasis underlie a host of emerging diseases, the calciumopathies. Cytosolic calcium signals originate either as extracellular calcium enters through plasma membrane ion channels or from the release of an intracellular store in the endoplasmic reticulum (ER) via inositol triphosphate receptor and ryanodine receptor channels. Therefore, to a large extent, calciumopathies represent a subset of the channelopathies, but include regulatory pathways and the mitochondria, the major intracellular calcium repository that dynamically participates with the ER stores in calcium signaling, thereby integrating cellular energy metabolism into these pathways, a process of emerging importance in the analysis of the neurodegenerative and neuropsychiatric diseases.

Molecular genetic analysis offers opportunities to advance our understanding of the nosological relationship between psychiatric diagnostic categories in general, and the mood and psychotic disorders in particular. The CACNA1C (alpha 1C subunit of the L-type voltage-gated calcium channel; SNP example rs1006737) gene encodes one subunit of a calcium channel. Results suggest that ion channelopathies may be involved in the pathogenesis of bipolar disorder, schizophrenia and autism with an overlap in their pathogenesis based upon disturbances in brain calcium channels.

CACNA1C encodes for the voltage-dependent calcium channel L-type, alpha 1c subunit. Gene variants in CACNA1 are associated with altered calcium gating and excessive neuronal depolarization. CACNA1 polymorphisms such as rs 10848635 and 1006737 are associated with increased risk of bipolar disease, and risk of SSRI induced suicidal ideation and changes in baseline agitation. Significant effects have been found of the G to A variant on total gray matter volume.

Psychiatric disease phenotypes, such as schizophrenia, bipolar disease, recurrent depression and autism, produce a constitutionally hyperexcitable neuronal state that is susceptible to periodic decompensations. The gene families and genetic lesions underlying these disorders may converge on CACNA1C, which encodes the voltage gated calcium channel which can be diagnostically evaluated for its role in schizophrenia, autism and bipolar disease.

Recent genetic studies found the A allele of the variant rs1006737 in the alpha 1C subunit of the L-type voltage-gated calcium channel (CACNA1C) gene to be overrepresented in patients suffering from bipolar disorder, schizophrenia or major depression.

Strong evidence of association at the polymorphism rs1006737 (within CACNA1C, the gene encoding the alpha-1C subunit of the L-type voltage-gated calcium channel) with the risk of bipolardisorder (BD) has recently been reported in a meta-analysis of three genome-wide association studies of BD. The bipolar risk allele CACNA1C rs1006737 conferred increased risk for schizophrenia and recurrent major depression with similar effect sizes to those previously observed in BD. These findings suggest some degree of overlap in the biological underpinnings of susceptibility to mental illness across the clinical spectrum of mood and psychotic disorders, and show that at least some loci can have a relatively general effect on susceptibility to diagnostic categories based upon alterations in calcium signaling.

The A allele in the variant described is associated with higher rates of mood disorder recurrence, treatment resistance, and paroxysmal psychotic states.

Agents claimed as having application to treat neuropsychiatric disorders associated with altered calcium signaling detected by CACNA1C diagnostic testing include: Flunarazine, candesartan and Hydroxyfasudil, Nimodipine, other L type voltage gated calcium channel blockers, Lithium, and anti convulsants including Valproic acid, Lamotrogine, Carbamezepine,

For example, Hydroxyfasudil may affect a protein kinase that serve to catalyze the phosphorylation of an amino acid side chain in various proteins. These enzymes control the majority of the signaling processes inside cells, thereby governing cell function, growth, differentiation and destruction (apoptosis) through reversible phosphorylation of the hydroxyl groups of serine, threonine and tyrosine residues in proteins.

A major signal transduction system utilized by cells is the RhoA-signalling pathway. RhoA is a small GTP binding protein that can be activated by several extracellular stimuli such as growth factor, hormones, mechanic stress, or osmotic change as well as high concentration of metabolite like glucose. RhoA activation involves GTP binding, conformation alteration, post-translational modification and activation of its intrinsic GTPase activity. Activated RhoA is capable of interacting with several effector proteins including ROCKs (Rho kinase) and transmit signals into cellular cytoplasm and nucleus.

Injury to the brain and spinal cord activates ROCKs, thereby causing neurodegeneration and inhibition of neuroregeneration like neurite growth and sprouting Inhibition of ROCKs results in induction of new axonal growth, axonal rewiring across lesions within the CNS, accelerated regeneration and enhanced functional recovery after acute neuronal injury.

1-(5-Isoquinolinesulfonyl)homopiperazine hydrochloride (“fasudil hydrochloride”) is commercially available under the trademark of “Eril Inj.” (manufactured by Asahi Kasei Pharma Corp.) and clinically used as an injection preparation for improving cerebrovascular spasm after a subarachnoid bleeding operation and an accompanying brain ischemia.

Hydroxy fasudil is a specific Rho-kinase inhibitor which suppresses the increase of Ca(2+) induced by Glutamate. The neuroprotective effect of hydroxy fasudil is attributed to repressing Glu excitotoxicity and calcium overload by inhibiting Ca(2+) release from Ca(2+) stores in neurons.

The use of fasudil as an orally bioavailble, novel antidepressant based upon its calcium mediated neuronal stabilization effects has been previously undisclosed.

Candesartan has been shown to modify the Angiotensin II response in tissue. Angiotensin II (Ang II) is a powerful signaling molecule in neurons and exerts some of its biological effects by modulating Ca(2+) currents. The physiological actions of Ang II in the brain, whether mediated by AT1 or AT2 receptors, involve changes in neuronal activity that are initiated by changes in the activity of membrane ionic currents and channels, intracellular signalling pathways couple neuronal AT1 and AT2 receptors to changes in the activity of membrane K+ and Ca2+ currents and channels.

Intracellular Ca2+ is known to play an important role in Ang II signaling in neurons and Ang II caused a rapid time-dependent increase in [Ca2+]I voltage-sensitive Ca2+ channels, which are the primary source of Ang II-induced increases in [Ca2+].

This observation leads to a previously undisclosed use of Candesartan or other ARB agents to treat neuropsychiatric disorders associated with abnormal calcium signaling in the brain. Candesartan, an AT(1) blocker, can improve conditions associated with abnormal Ca(2+) release mechanisms due to the observation that AT(1) receptor blockade protects neurons of cellular alterations typically associated with calcium mediated hyperexcitability. Therefore, prevention of these alterations by candesartan may present a useful and novel pharmacological strategy for the treatment of neuropsychiatric disorders associated with altered calcium signaling in the brain, such as mood disorders, autism, bipolar disease and schizophrenia.

A variety of A-II antagonists are, or will be, known to one skilled in the art. Subcutaneous or oral administration of the ARB candesartan inhibits brain as well as peripheral AT(1) receptors, indicating transport across the blood-brain barrier, making it the preferred embodiment of this invention because this invention applies to a novel use of this agent to treat disorders of the CNS.

Flunarizine is known as a nonspecific calcium channel blocker that has been used for decades for the treatment of migraine, vertigo, and cognitive deficits related to cerebrovascular disorders. Flunarizine also has dopamine D2 receptor blocking properties and was effective in animal models of predictive validity for antipsychotics. However, its clinical antipsychotic efficacy compared to haloperidol demonstrated no significant differences in PANSS overall score. It has a unique pharmacokinetic profile as an oral drug with long half-life (2-7 weeks).

The use of flunarazine in patients with mood disorders associated with cacna1c polymorphisms has been previously undisclosed.

In summary, results of this assay which indicate polymorphisms in the L type voltage gated calcium channel suggests a treatment or class of treatments which reduce excessive neuronal depolarization. These include mood stabilizers, Lithium, anti convulsants and specifically centrally acting calcium channel blockers such as Fasudil, Flunazarine, Nimodipine and Candesartan.

### Stress Response and the Hypothalamic—Pituitary Adrenal Axis Genes in Depression

FKBP5 and BDNF Genes

FKBP5 regulates the cortisol-binding affinity and nuclear translocation of the glucocorticoid receptor. FKBP5 is a glucocorticoid receptor-regulating co-chaperone of hsp-90 and plays a role in the regulation of the hypothalamic-pituitary-adrenocortical system and the pathophysiology of depression.

FK506 regulates glucocorticoid receptor (GR) sensitivity. When it is bound the FKBP5 receptor complex, cortisol binds with lower affinity and nuclear translocation of the receptor is less efficient. FKBP5 expression is induced by glucocorticoid receptor activation, which provides an ultra-short feedback loop for GR-sensitivity.

Changes in the hypothalamic-pituitary-adrenocortical (HPA) system are characteristic of depression. Because the effects of glucocorticoids are mediated by the glucocorticoid receptor (GR), and GR function is impaired in major depression, due to reduced GR-mediated negative feedback on the HPA axis. Antidepressants have direct effects on the GR, leading to enhanced GR function and increased GR expression.

Polymorphisms the gene encoding this co-chaperone have been shown to associate with differential up-regulation of FKBP5 following GR activation and differences in GR sensitivity and stress hormone system regulation. Alleles associated with enhanced expression of FKBP5 following GR activation, lead to an increased GR resistance and decreased efficiency of the negative feedback of the stress hormone axis. This results in a prolongation of stress hormone system activation following exposure to stress. This dysregulated stress response might be a risk factor for stress-related psychiatric disorders.

Various studies have identified single nucleotide polymorphisms (SNPs) in the FKBP5 gene associated with response to antidepressants, and one study found an association with diagnosis of depression. Polymorphisms at the FKBP5 locus have also been associated with increased recurrence risk of depressive episodes. A recent study showed that FKBP5 genotypes also moderated the risk of post-traumatic stress disorder (PTSD). Four single-nucleotide polymorphisms (SNPs) FKBP5, rs3800373, rs9296158, rs1360780, and rs9470080, were genotyped on the complete sample.

In fact, the same alleles are over-represented in individuals with major depression, bipolar disorder and post-traumatic stress disorder.

Individuals homozygous for the TT-genotype at one of the markers (rs1360780) reported more depressive episodes and responded better to antidepressant treatment.

The stress hormone-regulating hypothalamic-pituitary-adrenal (HPA) axis has been implicated in the causality as well as the treatment of depression. FKBP5, a glucocorticoid receptor-regulating cochaperone of hsp-90, has been implicated in maintaining the HPA, and in depression. For example, recurrence of depressive episodes was observed with single-nucleotide polymorphisms FKBP5. These single-nucleotide polymorphisms were also associated with increased intracellular FKBP5 protein expression, which triggers adaptive changes in glucocorticoid receptor.

Major depression is associated with reduced hippocampal volume linked to stress and high glucocorticoid secretion.

In animal models, pretreatment with candesartan profoundly modifies the response to stress. The ARB prevents the central sympathetic activation characteristic of isolation stress and abolishes the activation of the hypothalamic-pituitary-adrenal axis during isolation.

Angiotensin II, through AT(1) receptor stimulation, is a major stress hormone, and that ARBs, in addition to their antihypertensive effects, may be considered for the treatment of neuropsychiatric disorders associated with FKBP5 polymorphisms.

Long-term pretreatment with an angiotensin II AT1 antagonist blocks angiotensin II effects in brain and abolishes the hypothalamic-pituitary-adrenal responses to isolation stress. AT1 receptor blockade prevented the isolation-induced increase in brain AT1 receptors and decrease in AT2 binding in the locus coeruleus. In addition, pretreatment with candesartan increased the time spent in and the number of entries to open arms of the elevated plus-maze, measure of decreased anxiety.

Calcium/calmodulin (Ca2+/CaM)-dependent protein kinase II (CaMKII) couples increases in cellular Ca2+ to fundamental responses in excitable cells. CaMKII is activated by angiotensin II, providing evidence that calcium signaling in the brain is activated by angiotensin II. The Ang II-induced apoptotic cascade converges in a common pathway mediated by CaMKII activation which results in p38MAPK activation and apoptosis.

Conversely, it follows that Angiotensin II blockade results in attenuated brain calcium signaling. It follows that modulation of abnormal calcium signaling via an AT(1) inhibitor may provide a novel means to treat altered calcium signaling associated with polymorphisms FKBP5.

A-II antagonist candesartan: 1-(cyclohexyloxycarbonyloxy)ethyl-2-ethoxy-1-[[2′-(1H)-tetrazol-5-yl)biphen yl-4-yl]methyl]benzimidazole-7-carboxylate and the pharmaceutically acceptable salts thereof which are disclosed in U.S. Pat. No. 5,196,444, the disclosure of which is incorporated herein by reference are claimed as specific agents for individuals with FKBP5 polymorphisms.

The dose administered must be carefully adjusted according to age, weight and condition of the patient, as well as the route of administration, dosage form and regimen and the desired result.

A preferred oral dosage form, such as tablets or capsules, will contain candesartan the ARB inhibitor in an amount of from about 1 to about 500 mg, preferably from about 1 to about 100 mg, and more preferably from about 5 to about 50 mg.

Tianeptine is also claimed as a potential psychopharmacological intervention based upon FKBP5 polymorphisms. Like the serotonin transporter polymorphism, individuals with FKBP5 polymorphisms may be susceptible to heightened cortisol as an effect of stress. This elevated cortisol leads to inhibition of glutamate reuptake, excess glutamate mediated neurotoxicity and structural changes in the brain in areas critical to cognition and emotion. Tianeptine is recognized as a dual serotonin transporter and glutamate transporter agonist, thus simultaneously reducing excess serotonin and glutamate, thereby preventing stress mediated neurotoxicity.

### Serotonin Transporter Related Genes

Serotonin neurotransmitter transporters are the targets of various therapeutic agents used in the treatment of depression and anxiety. Specifically, the selective serotonin reuptake inhibitors, are the most widely prescribed agents for depression. The SSRI mechanism of action in depression is mediated by these agents acting as selective antagonists of the serotonin neurotransmitter transporter. Antagonists block uptake and prolong and/or enhance the action of serotonin. SSRI agents, drugs most widely used in depression, selectively block the reuptake of serotonin and result in increased serotonin in the synapse.

SLC6A4 5-HTTLPR (5-hydroxytryptamine Transporter Linked Polymorphic Region)

The serotonin transporter (5-HTT) is a high affinity carrier protein, localized to the plasma membrane of the presynaptic neuron. The role of 5-HTT is to remove serotonin (5-HT) from the synaptic cleft, resulting in serotonin reuptake into the presynaptic terminus. Elevated synaptic serotonin levels are associated with improved mood; thus the effectiveness of many antidepressant drugs (namely selective serotonin reuptake inhibitors, SSRIs) is thought to be due to their inhibition of the serotonin transporter, thereby reducing serotonin reuptake into the presynaptic terminus, and increasing serotonin availability in the synaptic cleft. In addition to mood improvement, elevated synaptic serotonin levels are also indirectly associated with a number of negative side effects including sleep disturbances, arousal, decreased gut motility, and sexual dysfunction.

### 5-HTTLPR Polymorphism

The short (S) allele results in 50% less expression of the active transporter protein as compared to the long (L) form. As these genetic differences in the 5-HTT affect both baseline serotonin levels and the availability of the transporter as a target for antidepressant therapy, they can affect the efficacy, of antidepressant therapy, the likelihood of side effects, and the nature and extent of depressive symptoms experienced. Studies have shown that compared to L/L patients, those homozygous for the short allele (S/S) are more likely to:

a) respond to antidepressant therapy more slowly,

b) experience adverse drug reactions (ADRs) during antidepressant therapy, and

c) develop major depression following adversity due to a poorer stress response.

In general, L/L individuals report a better and faster response to SSRI therapy than S/S patients. While these L/L individuals may demonstrate appropriate response to SSRI therapy in 2 to 4 weeks, individuals with the short allele (L/S or S/S) may respond to SSRI therapy much more slowly or may benefit from non-selective antidepressants.

In a meta analysis regarding the relationship of the serotonin transporter and depression, The SS genotype was significantly associated with an increased risk of MDD among Caucasian populations.

In addition to serotonin transporters being targets for anti depressant therapy, it is also recognized that assessment of serotonin transporter activity may be a useful biomarker in psychiatry. Various studies have demonstrated that patients with serotonin transporter short alleles are less likely to respond to SSRI therapy and are also more likely to experience treatment emergent side effects. The specific gene which is tested for, referred to as either the 5HTTLPR or SLC6A4, regulates the rate of serotonin metabolism. This gene controls a receptor located in the synaptic cleft. The receptor binds to serotonin and shuttles it back to the presynaptic neuron, terminating its activity at the post synaptic junction. The binding affinity of this receptor (referred to as SERT) is regulated by hereditary factors related to the length of an allele. Short alleles have reduced binding affinity effects on the serotonin transporter. Conversely, long alleles have better affinity, resulting in a more efficient reuptake process. Thus, the inherited short allele of the serotonin transporter results in more synaptic serotonin and the inherited tong allele leads to reduced serotonin in the synapse. The neurochemical consequences of possessing short alleles of the serotonin transporter results in increased synaptic serotonin, an effect that should be associated with better outcomes in antidepressant treatment based upon the conventional notion that increased synaptic serotonin is equated with better anti depressant response. However, in many studies the patients who are less likely to respond to serotonin agonist anti depressant therapy are precisely those who have a genetic predisposition to have relatively higher levels of serotonin in the synapse. Thus, results of large scale genomic studies which have correlated a percentage of patients who have depression associated with a short allele (and subsequently higher levels of synaptic serotonin), supports the notion that in a unique and previously unrecognized group of patients, there appears to be unique phenotype of depression characterized by higher, rather than lower, synaptic serotonin. It follows that in this unique subset of patients who are characterized by higher synaptic serotonin, the metabolic target should be to enhance serotonin reuptake as opposed to inhibiting serotonin reuptake.

Tianeptine has been described in French Patent Specification FR 2 104 728 as a new medicament for use in the treatment of psychoneurotic disorders. Furthermore, French Patent Specification FR 2 635 461 describes the use of tianeptine and compounds thereof in the treatment of stress. Tianeptine has a unique mechanism of action which is completely opposite drugs which are currently used for depression. Tianeptine no only activates serotonin reuptake into the synaptic ending but also activates its release from the ending into the synaptic cleft thus accelerating serotonin turnover rate in the synapse, a mechanism which is unique and opposite the majority of anti depressants in clinical use (such as the SSRI agents), which increase, rather than decrease synaptic levels of serotonin.

Tianeptine is a clinically used antidepressant that has drawn much attention, because this compound challenges traditional monoaminergic hypotheses of depression. It is now acknowledged that the antidepressant actions of tianeptine can be attributed to its particular neurobiological properties which are opposite those of traditional antidepressants, such as the SSRI class.

Acute treatment with tianeptine significantly enhances the levels of metabolites of 5-HT and 5-hydroxyindole acetic acid in the brain. In contrast to that found with inhibitors of the uptake of 5-HT such as the SSRIs, treatment with tianeptine markedly enhances the depletion of 5-HT. In vitro measurement of the uptake of 5-HT also confirms that tianeptine exerts opposite effects to those of classical SSRI antidepressants, since the in vivo administration of tianeptine induced a significant increase in the uptake of 5-HT in synapses. The fact that both inhibitors of the uptake of 5-HT (SSRIs) and tianeptine which, in contrast, enhances the in vivo uptake of 5-HT, are both potent and efficacious antidepressants, challenges the current hypothesis that SE reuptake is the exclusive mechanism of antidepressant response and that in subsets of patients, the opposite neurochemical effects—i.e., enhanced serotonin reuptake, may be the preferred mechanism to achieve an antidepressant response.

Described herein are novel methods and means for determining the genotype of the serotonin transport gene in order to selectively prescribe a treatment that is ideally coupled to patients with this specific genomic variation. In particular, described herein are methods for the use of tianeptine, of isomers thereof and of salts thereof, intended for the treatment of a specific subtype of depression associated with the short allele of the serotonin transporter.

general, described herein are methods of treating depression by determining the genotype of an individual patient's serotonin transporter (SERT), and prescribing a modulator of serotonin re-uptake and/or release based on the particular allele of that individual. The use of a genetic test in which a specific polymorphism of the serotonin transport gene is detected which informs the clinician that a patient likely has higher (rather than conventionally predicted tower) serotonin subsequently alter the decision to choose a SSRE instead of an SSRI.

For example, described herein are methods of treating an individual for depression by determining the individuals genotype for the serotonin transporter, and determining the appropriate prescription for a selective serotonin reuptake enhancer (SSRE) drug based on the genotype. Although the primary a selective serotonin reuptake enhancer (SSRE) drug described at the present time is tianeptine (Stablon, Coaxil, Tatinol), the methods described herein may be used with any appropriate a serotonin reuptake enhancer. Tianeptine is currently used as an antidepressant for the treatment or prophylaxis of depression in specific subtypes of depression. The methods described herein include the treatment of subjects exhibiting a particular genotype of the serotonin transporter with a selectively prescribed SSRE (e,g., tianeptine). Thus, the administration of an SSRE (such as tianeptine) is based upon the genotype. For example, the decision to prescribe and/or the dosage of a SSRE may be based upon the length of the patient's serotonin transporter allele. In some variations, patients with the short allele version of the transporter are selectively prescribed tianeptine. An alternative or supplemental treatment may be indicated in patents with longer alleles. For example, patient' with longer alleles may be prescribed serotonin reuptake inhibitors (e.g., SSRIs or other tricyclic compounds).

Thus, in the above example, a drug such as Tianeptine, which acts specifically as a serotonin reuptake enhancer, would be more appropriate because the metabolic and inherited state of the patient identifies a hyperserotonin state associated with depression.

The methods described herein are based on the recognition that by assessing genotypes (long vs. short alleles) of a polymorphism of the promoter region of the gene that encodes the serotonin transporter (5HTTLPR), one can identify persons who are more likely to respond to alternative anti-depressant therapies based upon unique and seemingly paradoxical effects on serotonin transporter. In these so-identified patients, a novel and previously undisclosed method of use for tianeptine is established based upon the expression and determination of the serotonin transport subtype. Thus, the methods described herein generally include the step of screening subjects for serotonin allele length, which may comprise determining the serotonin transporter gene promoter genotype (with respect to long and short alleles thereof) of a subject. The serotonin transporter gene promoter genotype may be used to indicate whether or not the subject will respond selectively to either a serotonin reuptake inhibitor, or more particularly, a serotonin reuptake agonist.

The methods described herein are particularly adapted to screening for tianeptine responsiveness based upon the expression of single nucleotide polymorphisms in the serotonin transporter. This invention discloses a novel indication for the use of tianeptine based upon the short allele of the serotonin transporter, and a mechanism intended to reduce, rather than enhance, synaptic serotonin.

In one particular embodiment, the method comprises determining the presence of two serotonin transporter gene promoter short alleles in a subject. If a subject is determined to have a depressed subtype characterized by higher synaptic serotonin (secondary to possession of the short allele of the serotonin transporter), tianeptine and/or enantiomers thereof is selectively prescribed, optionally in the form of pharmaceutically acceptable salts, shall be presented in pharmaceutical forms.

In addition to the dosage calibration by genotype, the dosage of the SSRE may vary according to the age and weight of the patient, the administration route, and the nature of the therapeutic indication and associated treatments. For example, in patients for whom tianeptine is indicated based on the genotype, the dose may range from 12.5 mg to 300 mg per dose or per administration. The number of administrations may also be modulated (e.g., 1×, 2×, 3×, 4× per day).

Any appropriate form of the SSRE may be used. For example, regarding tianeptine, bases that convert tianeptine or enantiomers thereof into salts may be used. The preferred salt of tianeptine is the sodium salt.

In some variations, an immediate-release form of the SSRE may be used. Immediate release may lead, in some subjects, to high blood peaks being obtained. A prolonged-release form may also be used. The prolonged-release form may make it possible to avoid these blood peaks and to obtain a uniform blood concentration in man. This may make it possible to reduce undesirable effects which may potentially occur by the “peak effect.” In one variation, a prolonged-release form of the sodium salt of tianeptine may be used to achieve a better therapeutic index in the treatment of anxiety and depression.

The dosage-release for tianeptine may be controlled in any appropriate manner. For example, a matrix tablet (as described in U.S. Pat. No. 5,888,542) that combines a polymer derived from cellulose and a calcium salt, may be used to compound the drug for controlled release of the active ingredient (e.g., tianeptine). This combination may be well-suited to the physicochemical characteristics of the sodium salt of tianeptine.

Controlled release (and particularly near-linear release) may make it possible to obtain a prolonged release of tianeptine leading to blood levels in the range between 50 and 300 ng/ml, up to 24 hours after administration of the tablet. As mentioned, in addition to the genotype, the unit dosage may be varied according to the age and the weight of the patient, and the nature and the seriousness of the condition. In general, dosage may range between 12.5 and 50 mg for a daily treatment in patients for whom the genotype screening suggests tianeptine is indicated.

Suitable routes for administration may include oral, parenteral, per- or trans-cutaneous, nasal, rectal, perlingual, sublingual tablets, glossettes, soft gelatin capsules, hard gelatin capsules, lozenges, suppositories, creams, ointments, dermal gels etc., and may include forms allowing the immediate release or the delayed and controlled release of the active ingredient.

A method for screening a subject for determining whether said subject is at an increased risk for depressed mood, said method comprising determining the subject's HTTLPR insertion/deletion polymorphism genotype within the serotonin transport (HTT) gene, wherein an LS heterozygote for the HTTLPR insertion/deletion polymorphism in the promoter region of the HTT gene has an increased risk for depressed mood. Subjects having the LS heterozygote for the insertion/deletion polymorphism in the promoter region of the serotonin transporter (HTT) gene have an increased risk of depression.

The short allele of the serotonin transporter has been suggested to be in epistasis with BDNF. For instance, the interaction between 5-HTTLPR and Val66Met polymorphisms significantly predicts dysfunctional thinking from before to after a standardized sad mood provocation. Cognitive reactivity increased among S/L(G) 5-HTTLPR homozygotes if they were also homozygous for the Val Val66Met allele, demonstrating biological epistasis between SLC6A4 and BDNF for predicting connectivity among neural structures involved in emotion regulation.

Some polymorphisms in the promoter region of the serotonin transporter gene (SLC6A4) are also involved in the pathogenesis/treatment of MDD; for instance, a single nucleotide substitution, rs25531 (A/G) in the serotonin transporter is also relevant. A variable number of tandem repeats (short (S) vs. long (L)) in the promoter region of the serotonin transporter gene (5-HTTLPR) and a functional variant of a single-nucleotide polymorphism (rs25531) in 5-HTTLPR have been associated with increased risk for major depressive disorder (MDD), this particular variant polymorphism rs 25531, referred to herein as L(g) carriers. In particular, relative to L/L homozygotes, S carriers and L(g)-allele carriers have a higher probability of developing depression after stressful life events. This is because individuals with the rs25531 polymorphism, despite having the long allele of the serotonin transporter, actually behave as if they possess the short allele. Based upon the functional consequences of this SLC6A4 polymorphisms, individuals with the rs25531 are predicted to respond in a similar fashion as those who actually possess the short allele of the transporter with reduced responsive effects to SSRI, more treatment emergent side effects and potentially better response to agents which enhance CaMKII mediated neurogenesis.

In summary, Tianeptine is claimed as a preferred agent in depressed individuals who express short variants of the serotonin transporter.

BDNF (rs6265) A>G Val66Met

Brain-derived neurotrophic factor is a member of the nerve growth factor family. It is induced by cortical neurons and is necessary neurogenesis and neuronal plasticity. BDNF has been shown to mediate the effects of repeated stress exposure and long term antidepressant treatment on neurogenesis and neuronal survival within the hippocampus. The BDNF Val66Met variant is associated with hippocampal dysfunction, anxiety, and depressive traits. Previous genetic work has identified a potential association between a Val66Met polymorphism in the BDNF gene and bipolar disorder. Meta-analysis based on all original published association studies between the Val66Met polymorphism and bipolar disorder up to May 2007 shows modest but statistically significant evidence for the association between the Val66Met polymorphism and bipolar disorder from 14 studies consisting of 4248 cases, 7080 control subjects and 858 nuclear families.

The BDNF gene may play a role in the regulation of stress response and in the biology of depression and the expression of brain-derived neurotrophic factor (BDNF) may be a downstream target of various antidepressants.

Exposure to stress causes dysfunctions in circuits connecting hippocampus and prefrontal cortex. BDNF is down-regulated after stress. Acute treatment with the antidepressants tianeptine reverses stress-induced down-regulation of BDNF. Tianeptine increases the phosphorylation of Ser831-GluA1. Psychological stress down-regulates a putative BDNF signaling cascade in the frontal cortex in a manner that is reversible by the antidepressant tianeptine. Thus agents which promote BDNF are novel mechanisms treat stress induced alterations in the limbic system.

Activation of AMPA receptors by agonists is thought to lead to a conformational change in the receptor causing rapid opening of the ion channel, which stimulates the phosphorylation of CAMK11/PKC sites and subsequently enhance BDNF expression.

Nefiracetam or Aniracetam are also agents that may be used to treat patients expressing the BDNF (rs6265) A>Ci Val66Met SNP.

A structural class of AMPA receptor positive modulators derived from artiracetam are called Ampakines. Aniracetam and Nefiracetam are neurological agents called ‘racetams’ that are analogs of piracetam. They are regarded as AMPA receptor potentiators and CaMKII agonists.

Small molecules that potentiate AMPA receptor show promise in the treatment of depression, a mechanism which also appears to be mediated by promoting BDNF via CaMKII pathways. Depression is associated with abnormal neuronal plasticity. AMPA receptors mediate transmission and plasticity at excitatory synapses in a manner which is positively regulated by phosphorylation at Ser831-GluR1, CaMKII/PKC site.

Aniracetam [1-(4-methoxybenzoyl)-2-pyrrolidinone] is AMPA receptor potentiator that preferentially slows AMPA receptor deactivation. AMPA receptor potentiators (ARPs), including aniracetam, antidepressant-like activity in preclinical tests. Unlike most currently used antidepressants. Interactions of aniracetam with proteins implicated in AMPA receptor trafficking and with scaffolding proteins appear to account for the enhanced membrane expression of AMPA receptors in the hippocampus after antidepressant treatment. The signal transduction and mote tar mechanisms underlying alpha-amino-3-hydroxy-5-methyl-4-isoxazole propiona (AMPA)-mediated neuroprotection evokes an accumulation of brain-derived neurotropic factor (BDNF) and enhance TrkB-tyrosine phosphorylation following the release of BDNF. AMPA also activate the downstream target of the phosphatidylinositol 3-kinase (PI3-K) pathway, Akt. The increase in BDNF gene expression appeared to be the downstream target of the PI3-K-dependent by AMPA agonists and Tianeptine (described above). Thus, AMPA receptors protect neurons through a mechanism involving BDNF release, TrkB receptor activation, and up-regulation of CaMKII which increase BDNF expression.

Olfactory bulbectomized (OBX) mice exhibit depressive-like behaviors. Chronic administration (1 mg/kg/day) of nefiracetam, a prototype cognitive enhancer, significantly improves depressive-like behaviors. Decreased calcium/calmocutin-dependent protein kinase II mediates the impairment of hippocampal long-term potentiation in the olfactory bulbectomized mice. Nefiracetam treatment (1 mg/kg/day) significantly elevated CaMKII in the amygdala, prefrontal cortex and hippocampal CA1 regions. Thus, CaMKII, activation mediated by nefiracetam treatment elicits an anti-depressive and cognition-enhancing.

Recommended aniracetam dosage is usually 1500 mg per day, taken in two 750 mg doses, one in the morning and one in the afternoon. Dose ranges can vary between 100 mg-5 grams. Recommended doses of Nefiracetam are 50-200 mg/day.

Tianeptine is claimed as an agent to treat depression associated with BDNF. The therapeutic potential of positive AMPA receptor modulators in the treatment of neurological and psychiatric diseases has been previously described, but its use in combination with Tianeptine, an atypical antidepressant with a similar mechanism of action, has been previously undisclosed.

Tianeptine increases BDNF expression in the amygdala, increases in neurotrophic factor expression that may participate in the enhancement of amygdala synaptic plasticity mediated by tianeptine. Preferred embodiments may include Tianeptine with Nefiracetam or Aniracetam in individuals with BDNF polymorphisms, associated with or without SERT ss allele subtype.

### Methylation Related Genes

Certain examples pertain to use of the MTHFR gene or related gene products for determining an individual's tendency to experience depression based upon the said individual's inability to methylate certain pathways involved in catecholamine synthesis and or degradation. In one example, diagnosis involves testing a sample obtained from a subject for the presence of a polymorphism in the MTHFR gene.

Certain examples pertain to use of the COMT gene or related gene products for determining an individual's risk of developing or maintaining an addiction based upon the individual's ability to metabolize or maintain normal levels of dopamine in the brain. In one example, diagnosis involves testing a sample obtained from a subject for the presence of a polymorphism in the COMT gene.

The 5,10-methylenetetrahydrofolate reductase (MTHFR) is a key enzyme for intracellular folate homeostasis and metabolism. Methylfolic acid, synthesized from folate by the enzyme MTHFR, is required for multiple biochemical effects in the brain. A primary role involves the synthesis of dopamine in the brain. Folic acid deficiency results in fatigue, reduced energy and depression. Low folate blood levels are correlated with depression and polymorphisms of the MTHFR gene are closely associated with risk of depression.

MTHFR irreversibly reduces 5-Methyltetrahydrofolate which is used to convert homocysteine to methionine by the enzyme methione synthetase. The c677T SNP of MTHFR has been associated with increased vulnerability to several conditions and symptoms including depression.

Nucleotide 677 in the MTHFR gene has two possibilities: C. or T. 677C (leading to alanine at amino acid 222); 677T (leading to a valine substitution at amino acid 222) encodes a thermolabite enzymes with reduced activity. The degree of enzyme thermolability (assessed as residual activity after heat inactivation) is much greater in 677TT individuals (18-22%) compared with 677CT (56%) and 677CC (66-67%).

Suitable MTHF gene polymorphisms include polymorphisms in the 5,10-methylenetetrahydrofolate. reductase (MTHFR) gene, including MTHFR C677T and its association with common psychiatric symptoms including fatigue and depressed mood. These symptoms are proposed to be due to hypomethylation of enzymes which breakdown dopamine through the COMT pathway. In this model, COMT is disinhibited due to low methylation status, resulting in increased dopamine breakdown.

For unipolar depression, the MTHFR C677T polymorphism has been we described and validated.

COMT is an enzyme involved in the degradation of dopamine, predominantly in the frontal cortex. Several polymorphisms in the COMT gene have been associated with poor cognition, diminished working memory, and increased anxiety as a consequence of altered dopamine catabolism. Suitable COMT gene polymorphisms include, e.g., a polymorphism in a Catechol O-methyltransferase (COMT) gene, the major enzyme determining prefrontal dopamine levels, which has a common functional polymorphism (val(158)met) that affects prefrontal function and working memory capacity and has also been associated with anxiety and emotional dysregulation. A single nucleotide polymorphism in the COMT (Val158/108Met) gene affects the concentration of dopamine in the prefrontal cortex.

The COMT 158val/val genotype confers a significant risk of worse response after 4-6 weeks of antidepressant treatment in patients with major depression. There is a negative influence of the higher activity COMT 158val/val genotype on antidepressant treatment response during the first 6 weeks of pharmacological treatment in major depression, possibly conferred by decreased dopamine availability. This finding suggests a potentially beneficial effect of an antidepressive add-on therapy with substances increasing dopamine availability tailored according to COMT val158met genotype by inhibiting excess COMT activity

Dopamine agonists which can be selectively employed to individuals with this COMT polymorphism include COMT inhibitors, MAO inhibitors, Methylfolate and S adenosyl methionine.

### Dopamine Based Single Nucleotide Polymorphisms

Dopamine receptor D2, also known as D2R, is a protein that, in humans, is encoded by the DRD2 gene. Of interest herein are DRD2 polymorphisms −141 c/d. Several lines of evidence suggest that antipsychotic drug efficacy is mediated by dopamine type 2 (D(2)) receptor blockade. Six studies reported results for the −141C Ins/Del polymorphism (total sample size: N=687) which indicated that the Del allele carrier is significantly associated with poorer antipsychotic drug response relative to the Ins/Ins genotype. These findings suggest that variation in the D(2) receptor gene can, in part, explain variation in the timing of clinical response to antipsychotics and higher risk of weight gain in deletion allele subtypes of the DRD2 gene.

Many antipsychotic medications carry a substantial liability for weight gain, and one mechanism common to all antipsychotics is binding to the dopamine D2 receptor. Carriers of the deletion allele showed significantly more weight gain after 6 weeks of treatment regardless of assigned medication. Thus, it is recommended that in patients who display the DRD2 del allele, either an alternative to a neuroleptic or a neuroleptic which had preferential antagonist effects at the 5HT2A>DRD2 be suggested.

### SNP Detection

As an example, a patient visits with a psychiatrist or other mental health worker. After taking a history, the health care worker obtains a small sample of tissue from the mouth and sends it to a specialized lab which is able to analyze the DNA through methods used to those skilled in the art. The lab determines over a brief period of time the results of the DNA test. As one example, the test indicates whether a patient has one of three subtypes related to the gene, referred to as either LL, LS, or SS (long/long long,/short, and short/short) Certain individuals will possess two short alleles. This indicates that the serotonin transporter is less efficient with the short allele than the version in the long allele. The value of this result is as an assessment of serotonin synaptic levels, a more specific serotonin modulation drug can be chosen.

Various real-time PCR methods can be used to detect SNPs, including, e.g., Taqman or molecular beacon-based assays (U.S. Pat. Nos. 5,210,015; 5,487,972; and PCT WO 95/13399) are useful in monitor for the presence of absence of a SNP. Many other SNP detection methods are known in the art, including, without limitation, DNA sequencing, sequencing by hybridization, dot blotting, oligonucleotide array (DNA Chip) hybridization analysis.

Applied Biosystems, Inc (Foster City, Calif.) has developed several aspects of SNP genotyping technology. In one well used protocol PCP amplification of a desired SNP region is conducted using targeting primers, including two allele-specific fluorogenic probes, each consisting of a different fluorescent reporter dye and a fluorescent quencher. Prior to PCR, proximity of the quencher to the fluorphore causes fluorescence resonance energy transfer (FRET), reducing the fluorescence from the reporter dye. During PCR, the 5′ nuclease activity of Taq digests the allele-specific probe bound to the region of the SNP, releasing the fluorescent dye from the quencher and allowing generation of a fluorescence signal.

Any tissue sample may be used for genotyping the polymorphisms described in this art, or for determining levels gene products, including but not limited to, blood, saliva, spinal fluid, brain biopsy, cultured cells, stool, urine, or frozen sections taken for histologic purposes. In certain examples, blood is obtained from a subject for assaying with respect to the mentioned polymorphisms. In an example, venous blood is obtained from a subject using standard venipuncture techniques. In another example, a buccal swab can be obtained for analysis.

In any of the variations described above, a report summarizing the findings/screenings, and providing therapeutic guidance or suggestions may be provided to the patient, the patient's physician, or both. In some variations the report is a written report (provided electronically or in paper) stating the results of screening for the polymorphism, and well as the proposed or alternative therapeutic information such as which drugs to propose for treatment of the individual given their specific genetic profile.

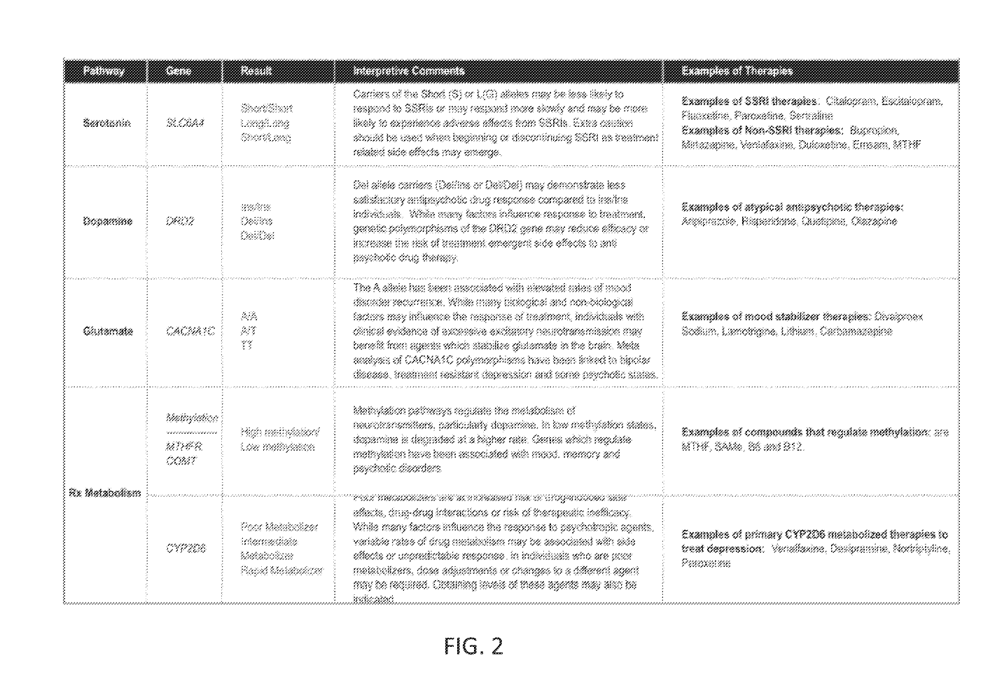

FIG. 2 is a table showing pathways tested (e.g., serotonin, dopamine, Glutamate, and drug metabolism), listing the genes, the polymorphism examined, and providing interpretive comments describing proposed therapeutic application of each of the examined and examples of therapies. In some variations, this report (customized to include an indication of an individual's results) is provided each time the test is run. One or all of the genes described herein may be included on the report; in some variations only a subset (e.g., one from each category) are included.

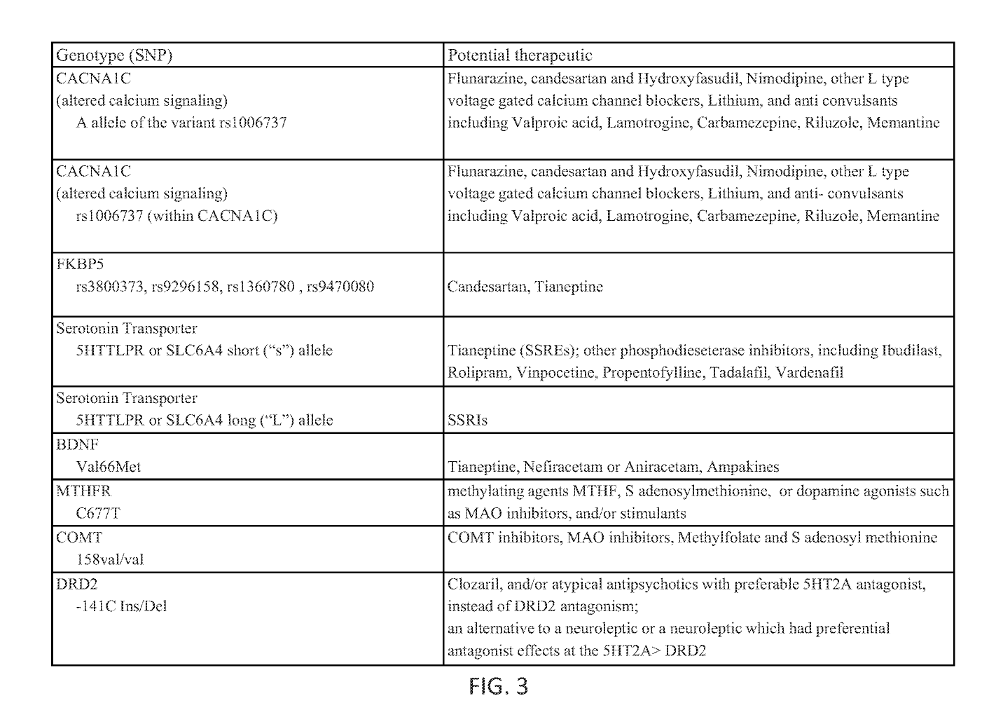

For example, a summary of the overall treatment recommendations based (in part) on one or more alleles of the genes described above. The table shown in FIG. 3 illustrates some of the therapeutic recommendations that may be provided based on the presence of each polymorphism. For example, tianeptine may be recommended in cases in which the s-allele of SERT is identified, and particularly SERT ss. BDNF polymorphisms may indicate the use of Tianeptine or agents of the chemical class racetams, such as Aniracetam or Nefiracetam. A polymorphism in CACNAIC may indicate the use of calcium channel blockers, Fasudil, Flunazarine, Nimodipine, Candesartan, etc. A polymorphism in FKBP5 may also suggest the use of tianeptine, or other phosphodiesterae inhibitors. The DRD2 del allele suggests Clozaril, and/or atypical antipsychotics with preferable 5HT2A antagonist, instead of DRD2 antagonism. Finally MTHF/COMT val/val polymorphism suggests the use of methylating agents MTHF, S adenosylmethionine, or dopamine agonists such as MAO inhibitors, and/or stimulants.

In general, the screens, assays, tests and reports described herein highlight key genetic loci forming a previously unrecognized epistatic group that are relevant to the treatment of depression (TRD). By determining a patient's genotype for the key genetic loci, as well as providing information specific the possible outcomes, the methods and reports described herein may enhance patient care.

What is claimed is:

1. A panel assay to determine the presence of SNPs that alter the time on or expression of a gene from each of the serotonin metabolism pathway, the dopamine metabolism pathway, the glutamate metabolism pathway, and the drug metabolism pathway.

2. The panel assay of claim 1, further comprising a report with one or more interpretive comments indicating the effect of any identified SNPs on the regulation of these pathways.

3. The panel assay of claim 1, further comprising an interpretive comment suggesting a treatment based on identified SNPs.

4. The panel assay of claim 1, wherein the SNP indicator indicates an SNP that alters the function or expression of the SERT gene in the serotonin metabolism pathway.

5. The panel assay of claim 1, wherein the SNP indicator indicates an SNP that alters the function or expression of the DRD2 genes in the dopamine metabolism pathway.

6. The panel assay of claim 1, wherein the SNP indicator indicates an SNP that alters the function or expression of the CACNA1C gene in the glutamate metabolism pathway.

7. The panel assay of claim 1, wherein the SNP indicator indicates an SNP that alters the function or expression of genes that regulate methylatoin and/or drug metabolism, including: MTHFR, COMT and/or CYP2D6.

8. The panel assay of claim 1, wherein the SNP indicators BDNF pathway comprise PCR-based assays.

9. The assay of claim 1, further comprising an interpretive comment suggesting a treatment based on identified SNPs.

10. The assay of claim 9, wherein the SNP indicator indicates an SNP that alters the function or expression of the SERT related genes in the serotonin metabolism pathway.

11. The assay of claim 9, wherein the SNP indicator indicates an SNP that alters the function or expression of the MTHF, or COMT genes in the methylation pathway.

12. The assay of claim 9, wherein the SNP indicator indicates an SNP that alters the function or expression of the CACNA1C genes in the glutamate metabolism pathway.

13. The assay of claim 9, wherein the SNP indicator indicates an SNP that alters the function or expression of the FKBP5 genes or BDNF in the hypothalamic pituitary adrenal axis.

14. A mood disorder panel assay to guide therapeutic treatment by determining the presence of SNPs that alter the function or expression of: the SERT gene; one or more of: DRD2, MTHF, COMT genes; one or more of: CACNA1C; and one or more of MTHFR, COMT and/or CYP2D6.

15. A mood disorder panel assay to determine the presence of SNPs that contribute to a mood disorder, the panel assay comprising:
a plurality of SNP indicators that collectively indicate the presence or absence of one or more SNP that alters the function or expression of a gene from each of the serotonin metabolism pathway, the dopamine metabolism pathway, the glutamate metabolism pathway, and the hypothalamic pituitary adrenal axis; and
an interpretive comment suggesting a treatment based on the identified SNPs.

16. A method of treating a patient for a mood disorder comprising:
determining the genotype of a polymorphism in each of the SERT, BDNF, CACNA1C, MTHFR/COMT and DRD2 genes;
advising a treatment based upon the results of said testing.

17. A method of treating a patient for a mood disorder comprising:
determining the genotype of a polymorphism in each of the SERT, BDNF, CACNA1C, MTHFR/COMT and DRD2 genes;
advising a treatment based upon the results of said testing wherein the treatment comprises:
prescribing at least one of tianeptine and other SSRE for patients having a SERT short allele;
prescribing at least one of Aniracetam and Nefiracetam for patients having the Val66Met form of BDNF;
prescribing at least one of a calcium channel antagonists, an L-type voltage gated calcium channel agonist, and a member of the ARB class of drugs, and Candesartan for patients having either the rs1006737 or the rs1006737 variant of CACNA1C;
prescribing at least one of a methylating agent, MTHF, S adenosylmethionine, a dopamine agonists, a MAO inhibitor, and a stimulant, for patients having either the C677T MTHFR variant or the 158val/val allele of the COMT gene; and
prescribing at least one of an atypical neuroleptics which preferentially inhibits 5HT2A over DRD2 and Clozaril for patients having the −141C Ins/Del.

18. A method of treating a patient for a mood disorder comprising:
determining the genotype of a polymorphism in each of the SERT, BDNF, CACNA1C, MTHFR/COMT and DRD2 genes;
providing a treatment based upon the results of said testing, wherein the treatment comprises:
prescribing at least one of tianeptine and other SSRE for patients having a SERT short allele;
prescribing at least one of Aniracetam and Nefiracetam for patients having the Val66Met form of BDNF;
prescribing at least one of a calcium channel antagonists, an L-type voltage gated calcium channel agonist, and a member of the ARB class of drugs, and Candesartan for patients having either the rs1006737 or the rs1006737 variant of CACNA1C;
prescribing at least one of a methylating agent, MTHF, S adenosylmethionine, a dopamine agonists, a MAO inhibitor, and a stimulant, for patients having either the C677T MTHFR variant or the 158val/val allele of the COMT gene; and
prescribing at least one of an atypical neuroleptics which preferentially inhibits 5HT2A over DRD2 and Clozaril for patients having the −141C Ins/Del.
